(19) United States
(12) Patent Application Publication
(10) Pub. No.: US 2024/0094047 A1
(43) Pub. Date: Mar. 21, 2024

(54) AGRICULTURAL MANAGEMENT SENSOR PLATFORM FOR FRUIT

(71) Applicant: Capture Actual Time Aus Pty Ltd, Panorama (AU)

(72) Inventors: Amanda MADER, Light Pass (AU); Craig HARRIS, Woodcroft (AU)

(73) Assignee: Capture Actual Time Aus Pty Ltd, Panorama (AU)

(21) Appl. No.: 17/947,236

(22) Filed: Sep. 19, 2022

(51) Int. Cl. G01G 19/18; G01G 23/37; G01N 27/22; G01N 33/02

(52) U.S. Cl. CPC G01G 19/18; G01G 23/3728; G01N 27/223; G01N 33/02

(57) ABSTRACT

An agricultural management sensor platform that comprises a load cell that measures the weight of the fruit or vegetable remotely in real-time. Other sensors such as temperature, moisture, humidity, and irrigation pressure are also implemented in the sensor platform. Data is captured in real-time and sent to an external server wherein the user can undertake further analysis.

### FIELD OF THE INVENTION

The present invention relates to an agriculture management sensor platform that can be used to monitor the growth of fruit weight.

### BACKGROUND TO THE INVENTION

As smart monitoring advances within the agricultural industry, it allows agricultural crop managers to monitor key parts of their crops to gather data to feed into their key decision-making processes.

One key crop in particular where there have been recent advancements with smart monitoring is grapes. Grapes are used for various purposes such as sultanas and wine, though a key area of interest for this patent is related to the use of grapes for producing wine.

For example, in relation to grapes for making wine, a winemaker will request/want a certain number of grapes to be harvested from a vineyard. Based on this the winemaker will invest in staff and materials to process the volume of grapes. Since the grapes are subjected to environmental conditions, the variation in the final grape yield is difficult to control and/or estimate. Therefore, if the end yield is less than the request, the winemaker will have invested in staff and materials that will not be used. If there is too much grape material produced, there will not be enough staff or material to handle the production and the excess number of grapes will be disposed of.

To better predict the final grape yield that will be produced, growers commonly cut grape bunches off the vines and weigh them, though this is a destructive method that requires a lot of grape bunches to be removed in order to get a suitable sample size. If multiple tests are undertaken it will result in a large number of grape bunches that are cut and destroyed, this adds up to a large loss of produce.

This patent overcomes the problems discussed above by providing a non-destructive and real-time continuous method to measure the weight of fruits or vegetables, in particular grapes for the wine industry.

### SUMMARY OF THE INVENTION

In a first aspect the invention there is a proposed sensor platform comprising of a loadcell sensor that is adapted to measure the weight of a fruit or a vegetable attached to a plant and wherein the loadcell sensor measures the weight data of the fruit or vegetable. Whilst the data may be stored locally typically it can be communicated to a remote server generally by wireless communication.

In preference the sensor platform further comprises temperature sensor wherein the temperature data around the fruit or vegetable is measured.

In preference the sensor platform further comprises a humidity sensor that measured humidity data.

In preference the sensor platform further comprises an irrigation water line pressure sensor that measures pressure data.

In preference the sensor platform further comprises a moisture sensor that measures moisture.

Preferably the data is stored on a memory module on the sensor platform.

Preferably the method of communicating to a remote server uses but is not limited to at one or more of the following methods: GSM, 3G, 4G, 5G, Wi-Fi, Sigfox, LoRa, Mesh, RS458, and Bluetooth.

Preferably the data is obtained in real-time and sent to the remote server that allows users to interface and undertake further analysis.

Preferably the weight data is used to predict the potential harvest weight of the fruit.

Preferably the weight data is used for evaluating and changing the irrigation plan for the plant.

Preferably a loadcell sensor is connected to a wire supporting the weight of the fruit.

It should be noted that any one of the aspects mentioned above may include any of the features of any of the other aspects mentioned above and may include any of the features of any of the embodiments described below as appropriate.

The object of this invention is to provide a system and method to address the above shortcomings or at least provide the public with a useful alternative.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows.

FIG. 1 is a schematic block diagram of an embodiment of the agricultural management sensor platform;

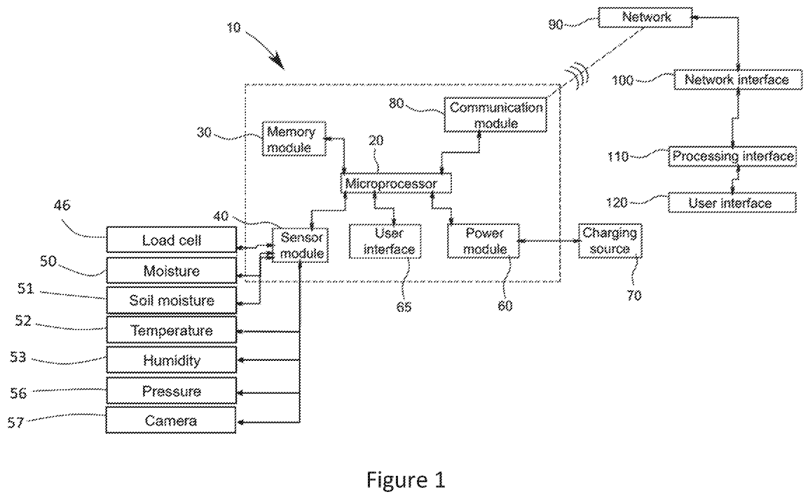

FIG. 2 is an illustration of the device; and

FIG. 3 is an illustration of the internal configuration of the device.

### DETAILED DESCRIPTION OF THE INVENTION

The unit 10 comprises of a microprocessor 20, a memory module 30, a sensor module 40, a power module 60, a user interface 65, a charging source 70, a communication module 80, network 90, network interface 100, processing interface 110, and a user interface 120.

The unit 10 firstly consists of a microprocessor 20. The microprocessor 20 can consist of but is not limited to analog inputs and outputs, digital inputs and outputs, TX, RX, ground, SCL and SDA, DAC, WKP.

Connected to the microprocessor 20 is a memory module 30, the memory module 30 can be but not limited to a SD card module wherein an SD card be inserted. The memory module 30 is used as storage for data, the SD card can be collected from the unit 10 if required. For example, if communications are not possible from a certain location, data can still be obtained from the SD card.

A sensor module 40 is connected to the microprocessor 20. The sensor module 20 is compatible with but is not limited to obtaining data from analog, digital, and 4-20 ma sensors. The sensor module 40 can transmit this information to the microprocessor 20 either by but not limited to a 120 protocol.

The device 10 also contains a power module 60 wherein it can obtain power from various sources and regulate the input power to the required voltage for the system 10. For example, a 12-volt battery can be connected to the power module 60, and the power module 60 will regulate the input power to the device 10. Though the voltage input and regulation for the device 10 is not a limitation and the input or required voltage to the system can be changed in accordance with the requirement of the unit 10. For example, the system voltage can be but is not limited to 1 to 240 volts.

The power module 60 can also be connected to an external charging source 70. The charging source 70 can be in the form of a solar panel wherein it captures solar energy and feeds power back into the power module 60, which in turn is used to charge the battery. The device 10 can also be connected to mains power if required.

The charging source 70 can also be in the form of a battery.

A communication module 80 is also connected to the microprocessor 20. The communication module 80 is capable of sending data from the microprocessor 20 to a desired network 90. The communication module can interact with the microprocessor 20 through an 120 protocol.

For example, the communication module 80 can communicate to a network using but not limited to Bluetooth, cellular, LoRa, mesh network, 2.4 gHz, RF, Zigbee, satellite, SDR, or Sigfox. Though it should be noted, the communication module 80 is not limited to these communication methods and can use any type of communication.

The communication module 80 can comprise of one or all of the above communication methods. The communication module 80 can send multiple messages out to various communication networks, or wherein if one network does not have a signal it will switch to another network. If there is no available means to communicate to a network it will record the data onto the memory module 30.

In most circumstances, it would be preferable to use a cellular network wherein a sim card is inserted into the communication module 80. The cellular network can be a category Ml, narrowband IoT cellular communication, 3G, 4G, and 5G.

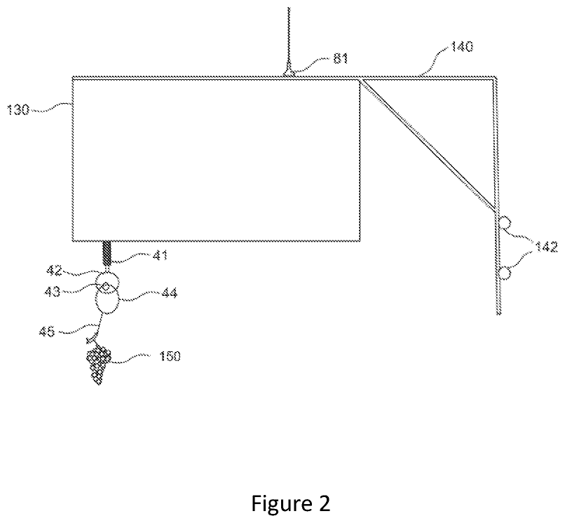

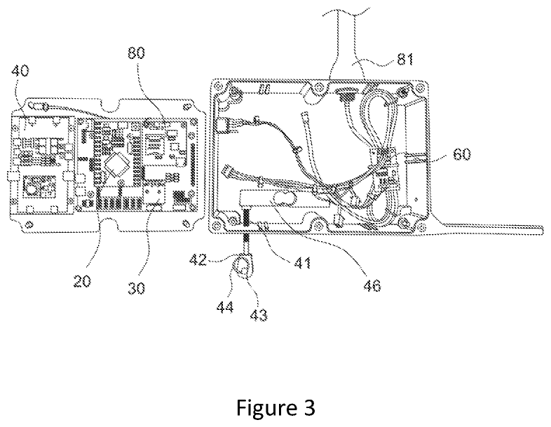

The antenna 81 can but is not limited to a gain of 3.8 dBi around 1.7 GHz to 2.7 Ghz, though an external antenna 81 can be used as shown in FIG. 2 and FIG. 3. It should be noted that the external antenna 81 is selected in accordance with the communication method used, therefore the type of antenna is not a limitation to the device.

A mesh network is wherein the sensor platform's function is to capture data and form a communication network. The distance the communication nodes can be from each other can be between 0.5 to 2 km. An example of a mesh network is wherein a sensor platform captures data and then passes that to another sensor platform, and wherein it is passed the data on to another sensor platform until it reaches its final destination.

The type of communication also determines the ability of the unit 10 to be programmed remotely. For example, if the unit 10 is using a cellular communication module 80 the unit can be programmed over the air if the user requires to update the system. Requests for data can also be sent from a user interface 120 to the unit 10 to obtain data upon request.

The unit 10 comprises of a sensor module 40. The sensor module 40 can connect to a variety of sensors 46, 50, 51, 52, 53, 56, 57. The sensor module 40 is the main link between the microprocessor 20 and the sensors. Though sensors can be connected directly to the microprocessor 20 if required.

The sensor module 40 can comprise of signal amplifiers and signal conditioners wherein the sensor data needs to be adjusted to ensure the readings that are captured are correct. The sensor module 40 can also provide the required power to a sensor wherein the microprocessor 20 voltage is lower than the sensor's required power. For example, the sensor module 40 can comprise of an analog to digital convertor.

A load cell 46 transducer can be connected to the sensor module 40. The load cell 46 is fixed to the unit cases 130 by a fixture on one end, and on the other end, a threaded rod 41 is attached. The threaded rod 41 protrudes out of the casing 130 from a hole in the bottom of the casing 130. It should be noted that the loadcell 46 is not limited to this location or mounting points and can be mounted externally and located remotely if required. For example, the load cell 46 can be located outside the box and connected with a wired or wireless to the sensor platform 10.

At the end of the threaded rod 41 is a U-shape hoop 42. A pin 43 travels through both ends of the U-shape hoop 42. The pin 43 is fixed in place by a ring 44, preventing the pin 43 from being removed from the U-shape hoop 42.

The ring 44 is attached to a wire 45. The wire 45 can then be attached to a branch supporting the weight of the fruit or vegetable 150, or to the fruit or vegetable 150 directly. The wire 45 can be but is not limited being 0.5 to 9 mm in diameter.

Alternatively, instead of using a wire 45 other forms of connecting the fruit or vegetable 150 can be used such as but not limited to string, chain, bag, or tape. The fruit or vegetable 150 can also be directly fastened to the ring 44, for example, a branch can be fed through the ring 44 or the ring can have a supporting article such as a shelf wherein the fruit or vegetable 150 hangs upon.

The fruit or vegetable 150 this the load cell transducer 46 can be used for but is not limited to are grapes, mangos, eggplant, strawberries, pears, apples, bananas, peaches, lemons, apricots, plums, berries, pawpaw, dragon fruit, or any other tree-based fruit and vegetable. It should be noted that this invention is not limited to the fruit and vegetables 150 listed above and can be applied to any fruit or vegetables or the plant itself.

As an example, the load cell transducer 46 captures the weight change of the grapes 150 as they grow over time, this is since the load cell 46 is connected to the grapes 150 and supporting close to but if not all the weight of the fruit 150. Preferably the load cell 46 will support all the weight of the fruit 150. As the grapes 150 start to increase in weight, the load cell 46 will start to strain wherein it provides a change in voltage that is captured by the sensor module 40. The load cell 46 can also capture weight loss wherein there is not enough water uptake by the plant or the plant is under stress. The fruit 150 does not need to be removed for the load cell 46 to be installed or to function.

Alternatively, if a different type of load cell 46 sensor is used such as a digital or 4-20 Ma sensor, the output signal will be captured and recorded by the sensor module 40. It should be noted that the invention is not limited to the type of load cell sensor 46 used, and any type of load cell sensor 46 can be used with this sensor platform.

The load cell 46 can also be connected to a supporting wire (not shown). A supporting wire is a wire wherein the plant is resting on or grows upon, this is commonly observed in vineyards where wires span between posts. The vines will grow along the supporting wires over time. Another example is a passion fruit vine, wherein the vines grow along with the supporting wires. The load cell 46 is attached to the wire, allowing the load cell 46 to capture changes in the weight along the wire. Changes in the weight along the wire can be attributed to the growth of the plant and the growth of the fruit or vegetable 150. This data can be used to determine the growth rate of a plant in addition to the fruit or vegetable 150 growth.

The number of load cell transducers 46 the unit 10 can comprise of can be but is not limited to 1 to 20. The type of load cell transducer 46 that can be used can be but is not limited to a straight bar, S-Type, disc, strain gauge, button, or compression load cell. Other forms of custom strain gauges can also be used wherein the supporting wire that is used to support the plant has a stain gauge built-in. The number of load cells 46 that can be used can be but is not limited to 1 to 20 sensors. The unit 10 will need to be modified to have additional input ports.

A moisture sensor 50 can be connected to the sensor module 40. The moisture sensor 50 is capable to capture the moisture content within the air that is around the fruit or vegetable 150 or the surface moisture content.

For example, the surface moisture content 50 sensor is capable to determine the percentage of hydration or moisture on the skin of the fruit or vegetable 150. The type of surface moisture sensor 50 used can be but is not limited to a printed copper trace sensor wherein it needs contact with the surface of the fruit or vegetable 150.

Soil moisture sensors 51 can also be used with the device 10. A soil moisture sensor is capable to capture the moisture levels within the soil below the plant. Soil moisture sensors 50 that can be used with the device can be but are not limited to capacitance, TDR, and neutron probe.

The depth of the soil moisture sensor 51 can be but is not limited to 1 cm to 10 m. The depth or position of the sensor is not limited and is determined by the user's requirement.

The distance of the soil moisture sensor 51 from the base of the plant can be but is not limited to 1 cm to 2 meters away from the tree.

The moisture sensor 50, 51 can be used to evaluate the effectiveness of irrigation or a rainfall event on the plants of interest. For example, a user may want to implement a new irrigation management plan to evaluate its effectiveness on fruit weight. As the user implements the new irrigation management plan, they will be able to evaluate changes in the moisture content in the soil, on the plant, or flesh of the fruit. This information can also be used to determine if a rainfall event was sufficient enough to water the plants or if additional irrigation will be required. This data can be combined with other sensor data, for example as the moisture content is maintained at a certain value, the weight data captured from the load cell 46 will start to increase in weight over time as the fruit or vegetable 150 grow. The amount of moisture sensors 50, 51 that can be used with the unit 10 can be but is not limited to 1 to 30 sensors.

A temperature sensor 52 can be used with the sensor module 40. A temperature sensor 52 can be in the form of negative temperature coefficient thermistors, resistance temperature detectors, thermocouples, semiconductor-based sensors, and infra-red non-contact sensors. The temperature sensors 52 can be positioned on the plant either on the outer surface of the fruit or vegetable 150, inside the fruit or vegetable 150 bunch, on the underside of the fruit or vegetable 150, along the branch, along the ground, or on the top surface of the plant. The amount of temperature sensors 52 that can be used but is not limited to 1 to 30.

Tracking the fruit 150 temperature is important when assessing the conversion of acids to sugars. For example, with grapes, grape maturation is critical for wine quality, this is measured by grape development (° Brix) and acidity (pH and Total Acidity). The speed of the ripening is measured by growing degree days (GDD). The temperature sensor 51 is capable to capture the fruit 150 temperature wherein it can be used to calculate the GDD or other means of calculating acid to sugar conversion. The temperature can also be used to determine the potential stress that the plant may be under, for example, high-temperature days can greatly stress a plant. Temperature data can also be linked with other sensors 46, 50, 51, 53, 56, 57 data to help predict potential plant health issues.

A humidity sensor 53 can be used with the unit 10. The type of humidity sensors that can be used may be but are not limited to capacitive, resistive, and thermal. The humidity sensor 53 data can be used to identify the potential present or risk of mould and mildew when combined with other sensors 46, 50, 51, 56, 57 data. The number of humidity sensors 53 that can be used may be but is not limited to 1 to 30 sensors.

A water pressure sensor 56 can be connected to the unit 10 to capture water pressure data in the irrigation network. The water pressure sensor 56 can be but is not limited to a differential or absolute pressure transducer.

Monitoring the irrigation pipe pressure with the water pressure sensors 56 can allow the user to detect any potential leaks, low-pressure zones, and blockages in the irrigation pipeline.

For example, if there is a leak in the irrigation line the pressure sensor 56 will detect an abnormal hydraulic grade line (HGL) pressure due to the water loss occurring near a pressure sensor 56, this may appear to be a lower static pressure in comparison to its calculated HGL pressure.

If there is a low-pressure zone within an irrigation network the pressure sensor 56 will capture the pressure information and provide this to the user. This will indicate to the user that the pressure in certain locations of the irrigation network is not being provided with enough water to effectively operate resulting in poor irrigation.

If blockages are present in the network, the pressure sensor 56 will capture a low-pressure reading since the volume of water travelling through a blockage is reduced. Though this is dependent on the location of the pressure sensor 56 in the network in accordance with the blockage location.

A camera 57 can also be attached to the unit 10. The camera 57 can capture a wide range of light frequencies ranging from FTIR to NIR spectrums. The camera 57 can be used to image the plants 160 and fruit or vegetables 150 over time to monitor growth, photosynthesis rates, and damage.

All the sensors 46, 50, 51, 52, 53, 56 data can be captured by the sensor module 40 and is sent to the microprocessor 20. The microprocessor 20 can then send the data to a remote server via the communication module 80.

The microprocessor 20 then saves the data onto the memory 30, which can be in the form of an SD card. The memory 30 unit can self-clean wherein after a set duration or number of records it will automatically clear the SD card memory and record over the old data.

The data 20 can be viewed on the user interface 65 that is on the outside of unit 10. This can be the form of a screen on the unit or a location wherein the user can connect a device such as a phone or a computer with a wired or wireless connection to obtain the data.

The screen for the user interface 65 can be a touch screen that can allow users to interface with and browse data sets as required.

The microprocessor 20 can also send the data to a remote server via the communications module 80.

The data is sent from the communication module 80 to a network 90, the network 90 then captures that information which may be sent to a network interface 100. A network interface 100 can be in the form of a remote server wherein the data is captured and stored.

The data can then be accessed on the network interface 100 by a processing interface 110. The processing interface 110 can consist of various functions wherein the data can be used to trigger certain actions. For example, if the temperature rises above a certain threshold the processing interface 110 will send an alert to the user. Alerts can be in the form of but are not limited to a SMS, email, phone app alert, social network post, or phone call.

The processing interface 110 can also undertake calculations on the raw data.

Calculations that can be undertaken may be but are not limited to a coefficient of variation (CV), potential error (PE), samples size, grape acid to sugar conversion, and average data. Though it should be noted that the calculations were undertaken are not limited to what has been discussed, and calculations that are known to someone skilled in the art of agricultural practices can be used, therefore the user will undertake calculations in accordance with their crop and practice methods.

The processing interface 110 is also capable to generate visual representations, such as plotting the data onto a map, forming graphs based on the data, forming tables from the data, and displaying pictures and video feeds from the camera 57.

The processing interface 110 can also analyse or undertake real-time calculations on the data as it is captured. For example, as the data flows into the processing interface 110 it is automatically averaged over a certain time that is set by the user, various other statistics can also be undertaken on the captured data such as standard deviations, mean, and any other form of statistic commonly used by someone skilled in the art.

The user interface 120 allows users to access the data that is captured by the sensors 46, 50, 51, 52, 53, 56, on the unit 10. The user interface 110 can either be through website access or through a software package capable to obtain data from the network interface 100 or processing interface 110.

The user interface 120 can also be set up to provide alerts to users wherein when a threshold is set by the user, for example, if the weight of a fruit or vegetable 150 goes below 100 grams a message will be sent to the user. The message can be in the form of and not limited to a text message, email, social media post, audible noise, or visual representation on the user interface or map visualisation. The user interface 120 can also be used to send messages to remote-controlled assets such as valves and pumps to remotely operate irrigation systems.

From the data obtained from the sensors 46, 50, 51, 52, 53, 56 in real-time from the field the user can implement new a decision. This data can also be forwarded to other services providers wherein they are capable to undertake further analysis and actions on the data. This may include artificial intelligence servers that can automatically operate remote-controlled valves in the field.

The device 10 is contained in a box 130. The box 130 can be any shape as required by the user, for example, but not limited to the box can be a cube or cylinder. The box 130 is connected to a bracket 140.

The bracket 140 can be made from but is not limited to steel, plastic, or wood. The bracket 140 can be attached to a post or the plant of interest with fixtures 142. As an example, if this device is used in a vineyard, the bracket 140 would be fixed to a post. A post can also be placed near the fruit 150 of interest, for example a starpicket or post can be fixed into the ground allow the bracket 140 to attach to something.

It can be attached with but is not limited to a fastener, string, cable tie, clip, chain, and bracket.

Below we will discuss various use examples for the device 10.

### Example 1

A winemaker requests a certain grape yield from a vineyard, this may be between 1 to 500 tons of grapes that need to be used in production. The vineyard manager can then assess the current weight of the vineyard by reviewing the sensor platform data 10, and implement an irrigation regime to achieve the desired bunch weight so they can meet the request from the winemaker's target weight. The weight data is captured by the load cell transducer 46. Over time the vineyard manager can continue to target key areas of the vineyard based on the sensor data 46, 50, 51, 52, 53, 56, this can include issuing new irrigation plans to ensure that the grapes meet the desired weight yield during harvest. The user will also be able to determine if shrinkage is occurring within the vineyard since the bunch weights 46 are reducing, they will be able to attribute this to low moisture 50, 51 contents or high bunch weight temperatures 52. The vineyard manager will also be able to assess that the weight of the grapes is increasing in accordance with an irrigation regime, therefore no further changes to an irrigation regime will need to be undertaken.

### Example 2

A rainfall event has taken place and the vineyard manager would like to determine what effect that has had on the grape bunch weight 150. Information such as grape bunch weight and moistures sensors 50, 51 can be analysed in real-time. This may reveal if certain parts of the vineyard received more rain than others. This data would be obtained from the moisture sensors 50, 51 and the weight sensors 46.

### Example 3

An irrigation plan is being developed for a fruit tree, the user now can review the growth of the fruit over time and better determine when the mangos will be ready for collection and organise the manpower required to pick the crop. Weight data 46 will increase over time as the fruit 150 is growing.

### Example 4

The vineyard manager has implanted an irrigation plan, the system provides an alert to the user that there is dropping pressure within a certain section of the irrigation system combined with low soil moisture content. The vineyard manager has identified that the pressure sensor 56 is downstream of a blockage in the pipe due to a partially shut valve.

### Example 5

The vineyard manager irrigation plan has been implemented. The device 10 discovers a pressure 56 reduction in the network is detected while irrigating, this is due to an undersized pump. This results in low bunch weights data 46 that are located around the low-pressure region and low soil moisture 51 contents.

### Example 6

The device 10 is monitoring the humidity 53 and grape bunch 150 temperature 52. The humidity 53 and the temperature 52 recognise that there is a high potential for mould and mildew to grow on the fruit 150. Therefore, the vineyard manager is capable to prepare in advance for mould and mildew. The vineyard manager is then able to implement a plan to mitigate the mould and mildew prior to it starting.

### LIST OF COMPONENTS

The drawings include the following integers.
10 Device
20 Microprocessor
30 Memory
40 Sensor
41 Screw
42 End mount
43 Pin
44 Ring
45 Wire
46 Load cell
50 Moisture
52 Temp and humidity
53 Humidity
56 Waterline pressure
57 Camera
60 Power module
65 user interface
70 Solar
80 Communications interface
81 Antenna
90 Network
100 Network interface
110 Processing interface
120 User interface
130 Case
140 Bracket
142 Fixtures
150 Fruit or vegetables

Further advantages and improvements may very well be made to the present invention without deviating from its scope. Although the invention has been shown and described in what is conceived to be the most practical and preferred embodiment, it is recognized that departures may be made therefrom within the scope of the invention, which is not to be limited to the details disclosed herein but is to be accorded the full scope of the claims so as to embrace any and all equivalent devices and apparatus. Any discussion of the prior art throughout the specification should in no way be considered as an admission that such prior art is widely known or forms part of the common general knowledge in this field.

In the present specification and claims (if any), the word “comprising” and its derivatives including “comprises” and “comprise” include each of the stated integers but does not exclude the inclusion of one or more further integers.

1. An agricultural management sensor platform comprising:
a sensor that captures weight data of a fruit or vegetable on a plant; and
wherein the weight data is recorded over time.

2. The agricultural management sensor platform in claim 1 wherein the sensor is a load cell transducer.

3. The agricultural management sensor platform in claim 1 further comprising; a temperature sensor, wherein temperature data around the fruit or vegetable is measured with the temperature sensor.

4. The agricultural management sensor platform in claim 1 further comprising; a humidity sensor, wherein humidity data around the fruit or vegetable is captured with the humidity sensor.

5. The agricultural management sensor platform in claim 1 further comprising; an irrigation water line pressure sensor, wherein pressure data is captured with the irrigation water line pressure sensor.

6. The agricultural management sensor platform in claim 1 further comprising; a moisture sensor, wherein moisture data is captured with the moisture sensor.

7. The agricultural management sensor platform in claim 1, further comprising: a memory module configured for storing data including the captured weight data.

8. The agricultural management sensor platform in claim 1 further comprising; a method of communicating to a remote server using at least one or more of GSM, 3G, 4G, 5G, Wi-Fi, Sigfox, LoRa, Mesh, RS458, and Bluetooth.

9. The agricultural management sensor platform in claim 1 wherein the data is obtained in real-time and sent to a server that allows users to interface and undertake further analysis.

10. The agricultural management sensor platform in claim 1 wherein the weight data is used to predict the potential harvest weight of the plant.

11. The agricultural management sensor platform in claim 1 wherein the weight data is used for evaluating and changing the irrigation of a plant.

12. The agricultural management sensor platform in claim 1 wherein the sensor is connected to a wire supporting the plant or fruit.
